(19) United States
(12) Patent Application Publication
(10) Pub. No.: US 2003/0180362 A1
(43) Pub. Date: Sep. 25, 2003

(54) Multi-stage oral drug controlled-release system

(75) Inventors: Jin Woo Park, Gangseo-gu (KR); Joon Ho Bae, Dongjak-gu (KR); Jung Ju Kim, Yongin-si (KR)

Correspondence Address: HESLIN ROTHENBERG FARLEY & MESITI PC, 5 COLUMBIA CIRCLE, ALBANY, NY, 12203, US

(73) Assignee: PACIFIC CORPORATION, Seoul (KR)

(21) Appl. No.: 10/357,821

(22) Filed: Feb. 3, 2003

(30) Foreign Application Priority Data
Feb. 1, 2002 (KR) 2002-5858

(51) Int. Cl. A61K 9/26; A61K 9/14

(52) U.S. Cl. 424/470

(57) ABSTRACT

A multi-stage oral drug controlled-release system is disclosed, as well as a preparation for maintaining the drug blood concentration at a desired level for a prolonged time. The system operates by releasing the drug at a constant rate through stepwise control of drug release following administration of the preparation. More specifically, the multi-stage oral drug controlled-release system involves the stepwise release of drug-containing granules from an inner matrix, which is surrounded by a coating or release-modifying layer. The granules contain an active drug and a carrier material in size of 0.1˜1 mm. The carrier material is hydrophobic when the drug has a water-solubility of 1 mg/ml or more, and is hydrophilic when the drug has a water-solubility of less than 1 mg/ml. The inner matrix, in which the drug-containing granules are embedded, is formed from swelling and erodible polymer(s) and swelling-regulating material(s). The release-modifying layer is composed of a hydrophobic release-modifying polymer, a hydrophilic release-modifying polymer, pH-dependent release-modifying polymer or mixtures thereof.

### CLAIM FOR FOREIGN PRIORITY

This application claims priority from Korean Patent Application No. 2002-5858, which was filed Feb. 1, 2002. The entire content of the prior application is incorporated herein by reference.

### FIELD OF THE INVENTION

The present invention relates to a novel oral drug delivery system for controlling drug release, a preparation for maintaining drug concentration in blood at a certain level for a prolonged time following administration by allowing the drug to be released at a constant rate through stepwise control of drug release.

### BACKGROUND OF THE INVENTION

Administration forms capable of controlling drug release become an important part of medication in terms of improved treatment effect, reduction of side effects and patient's convenience. Controlled-release of active drug provides many therapeutic advantages, the most important of which is that the blood level of the drug can be maintained for long time with minimal fluctuation. Thus, ensuring a constant rate of drug release from a preparation is an important aspect in controlled-release preparations. This may be accomplished by ensuring that an amount of the drug equivalent to that eliminated from the body, is continuously released from the preparation for absorption as it passes through the gastrointestinal tract.

In general, controlled-release preparations may be characterized as three types: (1) a dosage wherein active drug-containing particles (granules) are coated; (2) a dosage having an internal matrix typically based on polymers; and a dosage relying on osmotic pressure. Of these, the matrix form tablet has achieved the greatest popularity due mainly to its ease of manufacture. When compared with controlled-release tablets, due to their very small size and resulting large surface area, granules suffer the disadvantage of relatively fast disintegration, which leads to a short drug-release time in the body.

Matrix-based preparations release their drug via diffusion, and various techniques such as introducing a water-insoluble coating layer on the drug-containing matrix particles have been developed. In the case where the components of coating layer and the matrix are insoluble in bodily fluids, the diffusion of the drug is controlled by the components of coating layer or matrix. Here, drug is released by concentration gradient of the drug introduced as water slowly penetrates the preparation. This method will typically show a decline in the release rate at the last stage due to the gradual reduction of the concentration gradient and the gradual increase of the diffusion distance. Accordingly, the release rate of the drug cannot be consistently maintained at a constant level, and it gradually decreases as a function of time.

Thus, simple matrix tablets extend the period of drug release, while exhibiting an inherent limit of releasing drug by first order kinetics or at a rate of (time)^{0.5}. Methods to improve the consistency of release from matrix formulations have attempted to reduce initial drug release rate by: (1) introduction of a coating layer; (2) inducing zero-order release rate through a morphological approach to preparation; (3) a combination of these two methods; as well as (4) by allowing the diffusion distance to be reduced as a function of time through the use of erodible and swelling polymers as a main component of matrix.

The majority of the alterations to matrix preparations, however were for purposes other than stabilizing the release rate of the active drug. A prime example is the use of enteric coating, which delays release of the drug until the dosage unit reaches the colon. Two good examples of morphological approaches to controlled-release systems, involve a method of regulating the release area by introducing a hydrophilic or hydrophobic layer on each side of the drug-containing layer and a method of exposing a constant area of the coated tablet.

Matrix formulations mainly consist of an active drug and a biocompatible polymer. Polymer matrices that swell and erode typically consist of a swelling layer, a diffusion layer and an erosion layer. This preparation has the advantage of being able to regulate drug release rate at a fixed level based on the moving rates of the swelling layer and the erosion layer. However, using an erosive polymer also has the disadvantage that the release area deceases with time, leading to a typical matrix release mechanism pattern wherein the release rate decreases along with the reduction of release area. In an attempt to control this drug release pattern, a coating layer and a component capable of controlling swelling were introduced. U.S. Pat. No. 6,156,343 for example, discusses the ability to retard swelling and the initial release by use of polyvinyl alcohol as the matrix core material, along with the addition of salt to the preparation and the introduction of a coating layer.

In addition to a simple erodible polymeric matrix system, non-erodible preparations with a coating layer comprising a water-insoluble polymer such as lacquer have also been developed, although they also cause a time-dependent reduction of drug release. With respect to osmotic preparation, these suffer the disadvantageous of cost and complexity of the system.

Two examples of attempts to prevent the reduction of drug release over time, include German patent documents DE 1,767,765 (discloses multi-layered tablets, wherein each layer has a different concentration of drug) and DE 2,651,176 (discloses a tablet in which the drug concentration increases from the outer layer inward toward the tablet core. However, similar to the osmotic preparations, these tablets require special and complicated manufacturing techniques and facilities.

U.S. Pat. No. 4,252,786 designed a preparation in which the core of water-insoluble swelling polymer swells with penetration of water to lead to burst of coating layer. Such pulsitile drug release is desirable for improving bioavailability of a drug whose first pass effect can be saturated, and it was revealed that drug release from the preparation is less sensitive to pH value of GI tract. Such preparation can freely control the delay of initial drug release, yet, drug release after the burst of the coating layer, still, depends on concentration gradient of drug.

U.S. Pat. No. 4,610,870 (Jain et al.) discloses a coated tablet showing zero-order release rate. The core of this tablet includes hydroxypropylmethylcellulose and/or methylcellulose, one or more non-swellable binders and/or wax binders, one or more inert fillers or excipients, and one or more lubricant.

U.S. Pat. No. 4,252,786 by Weiss et al. resolved the rapid initial-release problem of swelling and erodible formulation by coating the swelling matrix core with a hydrophobic film coating layer capable of burst. Drug release in this preparation occurs via diffusion through initial non-damaged coating layer, and core expands by continuous penetration of external fluid, leading to burst of the coating layer. Thereafter, the swelling matrix core controls the drug release. Overall drug release is continuous based on such control of initial release, and zero-order release can be achieved.

Although the foregoing inventions resolved the problem of non-linear drug release that can occur in swelling and erodible matrix tablet by introducing a coating layer, the result is still a simple coated tablet, which fails to overcome the basic limitations of swelling and erodible matrices. In addition, such formulations are ineffective for prolonged release (e.g. over 24 hours) of a highly water-soluble drug.

U.S. Pat. Nos. 4,309,404 and 4,248,857 (DeNeale et al.) teach the use of carboxypolymethylene as the core substance and introduced the concept of seal coating and sugar coating the tablets. U.S. Pat. No. 4,309,405 (Guley et al.) discloses a similar formulation which uses a combination of hydroxypropylmethylcellulose or hydroxypropylcellulose and hydrophobic polymer as the core substance. These two formulations demonstrated zero-order release pattern over 12 hours, but this was only after a rapid initial drug release for the first hour.

U.S. Pat. No. 4,610,870 discloses a coated tablet showing a zero-order release pattern over an 8 to 12 hour period. The coating layer inhibits a rapid initial drug release, as it gradually disappears by swelling of the core layer, wherein the drug is then released with erosion of the core.

U.S. Pat. No. 5,464,633 discloses the use of a compressed layer rather than a coating layer on a swelling and erodible core matrix tablet in order to modify drug release rate. This concept prevents rapid initial drug release, and at the same time, endows a sustained release effect over prolonged time. While this formulation was an improvement over the inconvenience of the tablet coating methods, it has its own disadvantages in requiring special facilities and complicated calculations to determine the appropriate release area.

U.S. Pat. No. 6,083,532 compensated for pH dependent behavior of drug solubility by using a combination of pH dependent substance and pH-independent polymer as a constituent of core matrix. Such release-modifying attempts were to make the release uninfluenced by individual patient's physiological condition, and applied as means for maximizing drug action. Such preparations can be applied to only specific group of drugs with specific pH-dependency, and as external fluid penetrates continuously into inside of the matrix, it sensitively reacts to pH within the gastrointestinal tract, thus it is difficult to expect continuously steady drug release.

U.S. Pat. No. 4,610,870 teaches a mixture of hydroxypropylmethylcellulose and methylcellulose as a gel-forming substance, and introduced a coating layer consisting of hydrophilic and hydrophobic materials on the core tablet. Based on this attempt, a preparation was designed to release procaine hydrochloride by zero-order over an 8 to 12 hour period.

U.S. Pat. No. 6,068,859 discloses a controlled-release preparation of azithromycin where, in order to control time-dependent release of drug, the drug was dispersed and embedded in core matrix comprising four kinds of hydro-colloidal gel-forming substance. Drug release was induced by erosion of the matrix, and when needed, a coating layer was introduced. As another method, a mixture of coated particles and particles without coating layer was introduced into a single capsule or tablet to allow drug to be released via release channel formed through the uncoated particles. Such preparations attempted to achieve a steady continuous release rate by combining each portion with different characteristics such as multi-particulate system. While this was a significant improvement, control of each part and the mixing ratio thereof is necessary, and the preparation thus requires a significant amount of time and effort to successfully accomplish the required results.

WO 99/47128 relates to tablet or capsule as biphasic sustained release delivery system, where particles comprising hydrophilic drug and hydrophobic polymer are dispersed in hydrophilic polymer. This system is applied to drugs with high water-solubility, such as metformin hydrochloride, to lead to increased release time and increased transit time in upper gastrointestinal tract by swelling of the preparation. Though the sustained release is effectively accomplished by controlling drug diffusion via adequate application of discontinuous phase of hydrophilic and hydrophobic substance, still, depends on concentration gradient. Therefore, it shows disadvantage of dumping effect due to rapid initial release and time-dependent reduction of release rate. Therefore, it exhibits sustained release effect for about 10 hr in case of drug with high water-solubility, yet represents typical release profile for a matrix tablet, and thus not effective in terms of long term drug release for more than 24 hours and release rate control.

The foregoing conventional techniques experience difficulty in releasing drug at a constant rate for a prolonged time due to substantial problems, such as time-dependent reduction of drug release area and increase of diffusion distance. With regard to osmotic pressure preparations, zero-order release can be induced, but requires a complicated manufacturing process and significantly high manufacturing costs.

### SUMMARY OF THE INVENTION

The present invention provides an oral drug controlled-release preparation having a minimized solubility-limit for drug and which can release the drug at a constant rate for a prolonged period of time without the typical disadvantages of time-dependent reduction of drug release area and increase of diffusion distance, or complicated manufacturing processes and high manufacturing costs as seen with osmotic preparations.

### DETAILED DESCRIPTION OF THE INVENTION

The present invention provides a novel oral drug controlled-release delivery system and a preparation for maintaining a steady-state drug blood concentration level for a prolonged period of time by releasing the drug at a constant rate through stepwise control of drug release following administration of the preparation.

More specifically, the principals of the present invention provide a controlled-release oral preparation characterized by stepwise release of granules from a matrix, followed by release of the drug from the granules.

The preparation comprises:

(1) granules comprising a drug and a carrier material in a size of 0.1˜1 mm, wherein the carrier material is a hydrophobic material when the active drug has a water-solubility of 1 mg/ml or more, and is a hydrophilic material when the active drug has a water-solubility of less than 1 mg/ml;

(2) a matrix in which the granules are embedded, the matrix comprising swelling and erodible polymer(s) and swelling-regulating material(s); and

(3) a release-modifying layer comprising a hydrophobic release-modifying polymer, a hydrophilic release-modifying polymer, a pH-dependent release-modifying polymer or a mixture thereof.

In general, the term “very soluble” is applied to a water-solubility of 1 mg/ml or more and there is no upper limit of the solubility. The principals of the present invention apply to any drug whose water-solubility is 1 mg/ml or more, and accordingly, may be applied to a drug having a water-solubility of about 1 g/ml.

The principals of the present invention apply equally to drugs having a water-solubility of less than 1 mg/ml, and as with the “very soluble” drug, there is no lower limit of the solubility. The principals of the present invention thus apply to any drug whose water-solubility is less than 1 mg/ml, and accordingly, may be applied to a drug having a water-solubility of about 0.1 ng/ml.

Preferably, about 50 to 100% of the active drug is present in granules, while the remaining drug resides within the erodible and swelling matrix, the release-modifying layer.

The coated swelling-matrix oral preparation for controlled drug release, according to the present invention, consists of three components: (1) granules containing a drug; (2) a swelling and erodible matrix wherein the drug-containing granules are embedded; and (3) a coating layer surrounding the matrix. With regard to the mechanism of drug release from the preparation, the coating layer provides an initial lag-time for a specific period of time; such as, for example, for enteric preparations or preparations for release at other specific sites in the body. The coating layer also functions to inhibit the dumping effect of drug release and to raise drug stability under storage.

When the controlled-release preparation is exposed to body fluid, the coating layer disappears upon the swelling of the inner matrix, which then leads to active swelling and erosion of the matrix. Swelling and erosion of the matrix then leads to the controlled-release of the drug-containing granules embedded in the matrix, whereupon the drug is released in a controlled manner from the granules.

With conventional swelling matrix systems, the direct release of drug from the inner matrix leads to a tendency for a time-dependent decrease of drug release rate. In distinct contrast, the principals of the present invention provide that the drug within the granules is directly released into the matrix, and at the same time, the drug-containing granules are continuously released and drug is released from the granules, i.e., a multi-stage controlled-release. Accordingly, the drug release area increases with time due to accumulated granules, which compensate for the reduction of release rate caused by the reduction of surface area of the erodible matrix itself, and thereby results in a controlled, constant rate release of drug.

With regard to the drug-containing granules, these comprise an amount of the active drug and a carrier material, wherein the size of the granules is approximately 0.1˜1 mm. When the active drug has a water-solubility of 1 mg/ml or more, the carrier material is hydrophobic, and when the active drug has a water-solubility of less than 1 mg/ml, the carrier material is hydrophilic.

Preferably, when the active drug has a water-solubility of less than 1 mg/ml, the drug-containing granules will be prepared by solid dispersion; when the active drug has a water-solubility within the range from 1 mg/ml to 100 mg/ml, the drug-containing granules will be prepared by wet granulation, and when the active drug has water-solubility of 100 mg/ml or more, the drug-containing granules will be prepared by dispersing the drug in hydrophobic fusible materials and forming the granules therefrom.

Preferred hydrophobic materials for forming the granules for very soluble active drugs, include, but are not limited to one or more component selected from the group consisting of fatty acids, fatty acid esters, fatty acid alcohols, fatty acid mono-, di-, tri-glycerides, waxes, hydrogenated castor oil, hydrogenated vegetable oil and the like.

Examples of the fatty acid alcohols include cetostearyl alcohol, stearyl alcohol, lauryl alcohol, myristyl alcohol and the like. Examples of the fatty acid esters include glyceryl monostearate, glycerol monooleate, acetylated monoglyceride, tristearin, tripalmitin, cetyl ester wax, glyceryl palmitostearate, glyceryl behanate (Compritol 888 ATO™) and the like. Examples of waxes include beeswax, camauba wax, glyco wax, castor wax and the like.

With active drugs of less than 1 mg/ml water solubility, preferable hydrophilic carrier materials for forming the granules include polyalkylene glycol and carboxyvinyl hydrophilic polymer, or a mixture thereof. Such materials could include, for example, polyethyleneglycol with a molecular weight of 1,000-6,000, carbomer (Carbopol™), calcium carboxymethylcellulose and sodium carboxymethylcellulose.

The granules may further comprise other additives and excipients, such as, for example, lactose, starch, mannitol, saccharose, glucose, sorbitol, dibasic calcium phosphate dihydrate, anhydrous dibasic calcium phosphate, microcrystalline cellulose (Avicel™), gelatin, salt, polyvinylpyrrolidone, or any combination thereof. In addition the granules may optionally include cross-linked sodium carboxymethylcellulose or cross-linked polyvinylpyrrolidone, to facilitate accelerated disintegration of granules. To correct pH dependence of drug, the granules might contain an inorganic acid and its conjugate base, or an organic acid (such as citric acid and tartaric acid) and its conjugate base.

The granules are the component that controls the release and ultimate absorption of the drug. With hydrophilic drugs, the control is achieved by diffusion through a hydrophobic granule, while with hydrophobic drugs, control is achieved with a hydrophilic granule, wherein a hydration environment is established around the granules and the increased surface area improves wet ability of the drug to increase the water-solubility thereof.

The second element of the present invention is a matrix wherein the drug-containing granules are embedded. The matrix preferably comprises swelling and erodible polymer(s) and swelling-regulating material(s).

Where the matrix is desired to be a hydrogel matrix, it may comprise at least one agent selected from the group consisting of hydroxyalkylcellulose, hydroxypropylalkylcellulose, polyalkylene oxide, sodium alginate, povidone, polyvinyl alcohol and sodium carboxymethylcellulose. Preferably, the matrix will comprise at lease one of polyethylene oxide, hydroxypropylcellulose, hydroxypropylmethylcellulose, sodium alginate, povidone polyvinyl alcohol or sodium carboxymethyl cellulose.

The matrix may optionally include an adjuvant for formation of the swelling and erodible matrix, such as, for example, cross-linked sodium carboxymethylcellulose or cross-linked polyvinylpyrrolidone, lactose, starch, mannitol, saccharose, glucose, sorbitol, dibasic calcium phosphate dihydrate, anhydrous dibasic calcium phosphate, microcrystalline cellulose (Avicel™), gelatin, polyvinylpyrrolidone, magnesium stearate, stearic acid, sodium stearate, talc, sodium benzoate, boric acid and colloidal silica. The matrix may further contain a portion of the active drug.

Swelling-regulating materials are used to control the degree and velocity of swelling of the polymer, and examples of such materials may include cross-linked sodium carboxymethylcellulose and cross-linked polyvinylpyrrolidone, or a mixture thereof. Preferably the concentration of t\The swelling-regulating material will be about 1 to 10% by weight to the total weight of matrix. The swelling and erodible polymer of the core matrix provides, via swelling, a hydration environment around the granules dispersed within the matrix, and thus can function to raise the solubility of a hydrophobic drug within the granules. As a second function, the core matrix also serves as a secondary drug release control, by controlling the release of granules from the surface by erosion.

The third element of the preparation according to the present invention is a release-modifying layer, which preferably comprises at least one component selected from the group consisting of hydrophobic release-modifying polymers, hydrophilic release-modifying polymers and pH-dependent release-modifying polymers.

In the present context of release-modifying layer, the term “modifying” means that drug release from the preparation controlled or modified by the layer.

Preferred hydrophobic release-modifying polymers include one or more of ethylcellulose, shellac and ammonio methacrylate copolymer (Eudragit RS™ or Eudragit RL™).

As a material for forming the coating layer, a hydrophilic release-modifying polymer might be selected from the group consisting of hydroxyalkylcellulose and hydoxypropylalkylcellulose or a mixture thereof, as well as hydroxymethylcellulose, hydroxyethylcellulose, hydroxypropylcellulose, hydroxybutylcellulose, hydroxypentylcellulose, hydroxypropylmethylcellulose, hydroxypropylbutylcellulose and hydroxypropylpentylcellulose.

pH-dependent release-modifying polymers are generally utilized as an enteric coating, and may include, for example, hydroxyalkylcellulose phthalate, hydroxyalkylmethylcellulose phthalate, cellulose acetyl phthalate, sodium cellulose acetate phthalate, cellulose ester phthalate, cellulose ether phthalate and anionic copolymer of methacrylic acid and methyl or ethyl methacrylate (for example, Eudragit®-L and Eudragit®-S), as well as mixtures thereof.

The release modifying layer may further comprise a plasticizer, such as, for example, castor oil, hydrogenated castor oil, fatty acid, substituted triglycerides and glyceride, polyethylene glycol of molecular weight within range of 300 to 50,000 and its derivatives. The release modifying layer, or coating layer, functions as the primary drug release control and modifies zero-order release rate of the matrix core. Use of pH dependent or hydrophobic polymer coating enables the formation of a target-oriented system. When a plasticizer is optionally included, it will preferably be present in a ratio of 5 to 50% by weight of the coating substance.

Preferably, the release modifying layer will be about 1 to 20% by weight to the total weight of the matrix. In the preparation of the coating solution, water or an organic solvent such as, for example, methanol, ethanol, isopropanol, acetone, chloroform, dichloromethane, or a mixture thereof, may be used.

The oral drug controlled-release system of the present invention comprises granules containing a therapeutically effective amount of drug, a swelling and erodible polymer matrix in which the granules are embedded, and a coating layer surrounding the drug-containing core matrix.

Preferably, the drug-containing granules will comprise approximately 50 to 80% by weight to the total weight of the preparation.

Drugs that may be used in the formation of the preparation of the present invention include, but are in no way intended to be limited to:

therapeutic agents for aconuresis of oxybutynin, tolterodine and therapeutically equivalent salts thereof; calcium channel blockers of nifedipine, verapamil, isradipin, nilvadipin, flunarizine, nimodipine, diltiazem, nicardipine, nisoldipin, felodipin, amlodipin, cinanzin and pendilin and pharmaceutically acceptable derivatives thereof;

beta-adrenergic antagonists of propranolol, metoprolol and pharmaceutically acceptable derivatives thereof;

angiotensin-converting enzyme inhibitors of captopril, enalapril, ramipril, fosinopril, altiopril, benazepril, libenzapril, alacepril, cilazapril, cilazaprilat, perindopril, zofedopril, lisinopril, imidapril, spirapril, rentiapril, delapril, alindapril, indalapril, quinalapril and therapeutically equivalent salts thereof;

non-steroidal anti-inflammatory agents of ketorolac, ketoprofen, benoxaprofen, caprofen, flubiprofen, fenoprofen, suprofen, fenbufen, ibuprofen, indoprofen, naproxen, miroprofen, oxaprozine, pranoprofen, pirprofen, thiaprofenic acid, fluprofen, alminoprofen, bucloxic acid, alclofenac acematacin, aspirin, indomethacin, ibufenac, isoxepac, profenac, fentiazac, clidanac, oxpinac, sulindac, tolmetin, zomepirac, zidornetacin, tenclofenac, tiopinac, mefenamic acid, flufenamic acid, niflumic acid, meclofenamic acid, tolfenamic acid, diflufenisal, isoxicam, sudoxicam and therapeutically equivalent salts thereof;

therapeutic agents for respiratory disorders of theophylline, salbutamol, aminophylline, dextromethorphan, pseudoephedrine and therapeutically equivalent salts thereof;

analgesics of tramadol, acetaminophen, morphine, hydromorphone, oxycodone, propoxyphene and therapeutically equivalent salts thereof;

psychoneural drugs of fluoxetine, paroxetine, buspirone, bupropion, carmabazepine, carvidopa, levodopa, methylphenidate, trazodone, valproic acid, amitriptyline, carbamazepine, ergoloid, haloperidol, lorazepam and therapeutically equivalent salts thereof;

antibiotics of azithromycin dihydrate, cepha antibiotics, clarithromycin, doxycycline, nitrofurantonin and therapeutically equivalent salts thereof,

antihyperlipidemic agent of bezafibrate, fenofibrate, ethofibrate, lovastatin and therapeutically equivalent salts thereof; antidiabetic agent of glyburide, glipizide, metformin and therapeutically equivalent salts thereof; as well as

cyclobenzaprin, favotidin, nizatidine, propafenone, clonazepam, hyoscyamine, diphenhydramine, olistat, doxazosin and therapeutically equivalent salts thereof.

With a water soluble active drug, the granules will preferably be prepared by wet granulation. For example, a drug, substance forming the granules as described above and at least one kind of additives are mixed and combined by adding binder solution comprising hydrophilic polymer and water or organic solvent such as denatured anhydrous ethanol as granulating fluid. Granulating fluid is added until wet mixture is formed and then the wet mixture is passed through 6˜18 mesh sieve. This is dried in an oven at 24 to 60° C. for 12 to 24 hours. The dried granules are screened with 10˜24 mesh sieve.

With a drug having a water-solubility of 50 mg/ml or more, for effective release-delay, the drug particles may be covered with a hydrophobic substance by melt-granulation. At a temperature of at least melting point of delivery system component, drug and other additives are mixed, dispersed and slowly cooled to obtain solid body of the delivery system, and granules are obtained by pulverization and screening.

With a hydrophobic active drug, it is preferable that the drug/granule component described above and at least one additive are admixed, melted at melting point of the granule component to obtain solid dispersion. For example, granule-forming additives are added to the formed solid dispersion until granules are formed. The granules are screened through 6˜18 mesh sieve, and then dried in an oven at 24 to 60° C. for 12 to 24 hours. The dried granules are screened with 10˜24 mesh sieve. Granules prepared as described above are mixed with swelling and erodible polymer and at least one additive forming matrix. Lubricant is added to the mixture and the final mixture is prepared into compressed tablet of core matrix without coating layer. The coating layer is preferably formed by using a hydrophobic polymer, a hydrophilic polymer or enteric/pH dependent substances, alone or in combination. At least one polymer for the formation of coating layer and plasticizer is made ready in a form dispersed in water or organic solvent and then the dispersion solution is sprayed on the core matrix prepared as above. Coated tablet is finally dried in an oven at 40 to 50° C. For stability and color of preparation, seal coating can be conducted. In order to allow drug concentration to rapidly reach effective blood level, 1 to 20% of drug can be directly contained within the coating layer.

The multi-stage oral drug controlled system involves drug release through the course of three individual steps:

1. First, the coating or release-modifying layer exhibits intentional release-delaying effect over a certain amount of time. When the coating layer comprises a single hydrophilic polymer, the overall release profile is not influenced and release pattern of the core matrix itself is maintained, leading to maintenance of zero-order release profile over an 8 to 24 hour period. When the coating layer comprises a mixture of a hydrophilic or enteric polymer and a hydrophobic polymer, after release-delay over a certain amount of time is maintained, as external fluid penetrates through pores formed by dissolution of the hydrophilic or enteric polymer and the hydrophilic plasticizer, allowing the penetrating fluid to initiate swelling of the core matrix. Swelling pressure of the core matrix causes the coating layer to disappear and zero-order release of drug occurs. When the coating is an enteric polymer, there is no drug release below pH 4.0, but instead, at pH 4.0 or more, release starts with loss of the coating layer.

2. Second, upon the disintegration and dissolution of the coating layer, the core matrix actively begins to swell, leading to the establishment of a hydration environment around the granules embedded in the matrix. As erosion of the matrix begins from the surface as the matrix swells, the drug-containing granules are released at a constant rate.

Preferably, as erosion of the matrix occurs, 0 to 20% of the total number of drug-containing granules are released within 4 hours, 0 to 50% are released within 8 hours, 0 to 70% are released within 16 hours, and most preferably, 0 to 100% are released within 24 hours.

3. As the third and final step, the active drug is released by diffusion through pores that have been formed within the granules, and by the osmotic pressure difference with the external body fluid.

The drug release pattern of the core matrix itself maintains zero-order release, and the introduction of the coating layer brings delay over a certain amount of time to lead to intentional appearance of biphasic zero-order release pattern. Release rate can be controlled in various ways, such as by altering the ratio of granules contained in the system, by altering the amount of swelling polymer, by altering the ratio of swelling matrix to granules, and by altering the ratio and amount of hydrophobic, hydrophilic or enteric polymer forming the coating layer.

The system prepared according to the present invention is an oral multi-stage controlled-release system and suitable for designing an oral drug delivery system for once or twice a day administration. The preparation provides controlled-release over an extended period of time, and may target a specific site for the drug's therapeutic purpose. Drug is released from granules that are released from the matrix by swelling and erosion, and accumulated released-granules allow surface area for drug release to be maintained at a constant level. Thus, this compensates the decrease of drug release rate according to reduction of surface area by erosion of matrix, leading to prolonged drug release at constant rate. Maintaining of zero-order release rate enables blood level of drug to be kept at a steady level for a long time.

### EXAMPLES

The principals of the present invention are illustrated through the following examples, which are non-limiting to the scope of the invention. Various changes in the examples will be apparent to the person of skill in the art, and all such variations are included within the scope of the claims that ultimately follow.

### Examples 1˜5

Preparations of Core Matrix Tablet Containing Oxybutynin

Oxybutynin, glyceryl behanate, solubilizer, binder, release-regulating agent and inert diluents were mixed for 10 minutes at dry state. The mixture, after water was added, was granulated for 5 minutes. The granules thus formed were screened through 18-mesh sieve and dried in an oven at 24 to 40° C. for 12 to 24 hours. The dried granules were screened with 20-mesh sieve.

Hydroxypropylmethylcellulose, binders, swelling-regulating agent and diluents were added to the screened granules, and then they were mixed for 10 minutes. Finally, lubricant was added to them, and then they were mixed for 5 minutes. The mixture was compressed to prepare tablets. Table 1 provides the ingredients of the core matrix tablet.

TABLE 1
Compositions of core matrix tablet containing oxybutynin

| Ingredient (mg) | Example 1 | Example 2 | Example 3 | Example 4 | Example 5 |
|---|---|---|---|---|---|
| Oxybutynin hydrochloride | 5 | 5 | 5 | 5 | 5 |
| Glyceryl behanate | 10 | 10 | 20 | 15 | 15 |
| Dibasic calcium phosphate dihydrate | 35.9 | 45.9 | 55.9 | 56.85 | 28.425 |
| Lactose | — | — | — | — | 28.425 |
| Sodium chloride | — | — | — | 17.63 | 17.63 |
| Sodium lauryl sulfate | 0.1 | 0.1 | 0.1 | 0.15 | 0.15 |
| Povidone | 6 | 6 | 6 | 9 | 9 |
| Cross-linked sodium carboxymethylcellulose | — | — | — | — | 15 |
| Hydroxypropylmethyl cellulose | 40 | 30 | 20 | 45 | 30 |
| Magnesium stearate | 3 | 3 | 3 | 1.5 | 1.5 |
| Total | 100 | 100 | 100 | 150 | 150 |

### Experimental Example 1

Dissolution Test for the Preparations of Examples 1˜5

Release profile of core matrix tablet prepared in said Examples 1-5 was determined by USP dissolution test method under conditions of simulated intestinal fluid (fluid II, pH 6.8), paddle type II and 50 rpm/900 ml and dissolution level according to time was measured. The results are shown as dissolution percentage as function of time in Table 2.

TABLE 2
Dissolution Percentage

| Time (hr) | Example 1 | Example 2 | Example 3 | Example 4 | Example 5 |
|---|---|---|---|---|---|
| 0 | 0.00 | 0.00 | 0.00 | 0.00 | 0.00 |
| 1 | 11.03 | 14.47 | 10.51 | 4.78 | 15.27 |
| 2 | 10.74 | 18.56 | 15.51 | 10.29 | 32.75 |
| 3 | 13.53 | 20.30 | 14.81 | 16.01 | 41.93 |
| 4 | 14.18 | 25.22 | 20.77 | 20.00 | 48.53 |
| 6 | 17.07 | 31.54 | 28.14 | 30.65 | 58.80 |
| 8 | 24.04 | 40.52 | 37.91 | 38.86 | 62.73 |
| 10 | 29.81 | 48.68 | 45.35 | 46.23 | 68.64 |
| 12 | 36.70 | 58.42 | 43.76 | 53.48 | 72.06 |
| 24 | 68.74 | 84.54 | 72.98 | 91.73 | 93.01 |

Based on the dissolution test result for the controlled-release preparation of the present invention obtained in Examples 1-5, it was confirmed that various controlled-release patterns of oxybutynin could be obtained by the core matrix tablet itself, and the release rate could be controlled by regulating the content of swelling and erodible polymer and glyceryl behanate. Example 4 represents zero-order release pattern over 24 hours, and Example 5 shows that the release pattern is affected by the content of swelling-regulating material contained in the matrix.

### Examples 6 and 7

Preparations of Core Matrix Tablet Containing Oxybutynin

Oxybutynin, glyceryl behanate, solubilizer, binder, release-regulating agent and inert diluents were mixed for 10 minutes at dry state. The mixture, after water was added, was granulated for 5 minutes. The granules thus formed were screened through 18-mesh sieve and dried in an oven at 24 to 40° C. for 12 to 24 hours. The dried granules were screened with 20-mesh sieve. Polyethylene oxide, binders, swelling-regulating agent and diluents were added to the screened granules, and then they were mixed for 10 minutes. Finally, lubricant was added to them, and then they were mixed for 5 minutes. The mixture was compressed to prepare tablets. Table 3 represents the ingredients of the core matrix tablet.

TABLE 3
Compositions of core matrix tablet containing oxybutynin

| Ingredient (mg) | Example 6 | Example 7 |
|---|---|---|
| Oxybutynin hydrochloride | 5 | 5 |
| Hydrogenated castor oil | 5 | 15 |
| Dibasic calcium phosphate dihydrate | 65 | 55 |
| Sodium chloride | 17.85 | 17.85 |
| Sodium lauryl sulfate | 0.15 | 0.15 |
| Povidone | 9 | 9 |
| Polyethylene oxide | 45 | 45 |
| Magnesium stearate | 3 | 3 |
| Total | 150 | 150 |

### Experimental Example 2

Dissolution Test for the Preparations of Examples 6 and 7

Release profiles of the core matrix tablets prepared in said Examples 6 and 7 were determined by USP dissolution test apparatus under conditions of simulated intestinal fluid (fluid II, pH 6.8), paddle type II and 50 rpm/900 ml and dissolution level according to time was measured. The result was represented by dissolution percentage as function of time in Table 4.

TABLE 4
Dissolution Percentage

| Time (hr) | Example 6 | Example 7 |
|---|---|---|
| 0 | 0.00 | 0.00 |
| 1 | 5.57 | 3.11 |
| 2 | 10.26 | 4.98 |
| 3 | 10.75 | 6.44 |
| 4 | 15.67 | 8.75 |
| 6 | 24.20 | 14.86 |
| 8 | 60.99 | 49.38 |
| 18 | 67.38 | 59.29 |
| 20 | 67.72 | 62.02 |
| 24 | 71.30 | 66.00 |

### Examples 8-10

Coating of Core Matrix Tablet Containing Oxybutynin

The core matrix tablet prepared in said Example 2 was coated with a mixture of hydrophilic release-modifying polymer and hydrophobic release-modifying polymer, more specifically, hydroxypropylmethylcellulose and ethylcellulose. Coating solution was prepared according to the composition given in Table 5. Spray coating was carried out in pan coater, and then the products were dried in oven at 40 to 50° C. for 12 to 24 hours.

TABLE 5
Coating solution composition

| Components (%) | Example 8 | Example 9 | Example 10 |
|---|---|---|---|
| Hydroxypropylmethylcellulose | 5.4 | 4.8 | 4.2 |
| Ethylcellulose | 0.6 | 1.2 | 1.8 |
| Castor oil | 0.7 | 0.7 | 0.7 |
| Ethanol | 46.7 | 46.7 | 46.7 |
| Methylene chloride | 46.7 | 46.7 | 46.7 |
| Coating %* | 3 | 3 | 3 |

*Coating degree to the weight of uncoated core matrix tablet is represented by %.

### Experimental Example 3

Dissolution Test for the Preparations of Examples 8˜10

Release profiles of the coated core matrix tablets prepared in said Examples 8-10 were determined by USP dissolution test apparatus under conditions of pH 4.0 solution, paddle type II and 50 rpm/900 ml and time-dependent dissolution level was measured. The result was represented by dissolution percentage as function of time in Table 6.

TABLE 6
Dissolution Percentage

| Time (hr) | Example 8 | Example 9 | Example 10 |
|---|---|---|---|
| 0 | 0.00 | 0.00 | 0.00 |
| 1 | 6.16 | 6.07 | 3.74 |
| 2 | 11.53 | 10.67 | 7.07 |
| 3 | 17.28 | 16.01 | 10.59 |
| 4 | 24.66 | 19.82 | 13.69 |
| 6 | 34.47 | 27.63 | 20.04 |
| 8 | 45.13 | 34.60 | 27.23 |
| 10 | 54.51 | 41.98 | 31.46 |
| 12 | 63.67 | 50.11 | 37.56 |
| 24 | 100.72 | 85.25 | 69.06 |

The dissolution test results for the coated core matrix of Examples 8 to 10 reveal that drug release rate of core matrix showing zero-order release pattern can be regulated by relative content of hydrophobic release-modifying substance contained in the coating layer.

### Examples 11 and 12

Coating of Core Matrix Tablet Containing Oxybutynin

The core matrix tablets prepared by said Examples 4 and 5 were coated with a mixture of hydrophobic release-modifying polymer and pore-forming substance, i.e. ethylcellulose and polyethyleneglycol (MW 300). Coating solution was prepared according to the composition given in Table 7. Spray coating was carried out in pan coater, and then the products were dried in oven at 40 to 50° C. for 12 to 24 hours.

TABLE 7
Coating solution composition

| Components (%) | Example 11 | Example 12 |
|---|---|---|
| Ethylcellulose | 7.0 | 7.0 |
| Polyethylene glycol (MW: 300) | 2.8 | 2.8 |
| Ethanol | 90.2 | 90.2 |
| Coating %* | 1.0 | 1.0 |

*Degree of coating to the weight of uncoated core matrix tablet is represented by %

### Experimental Example 4

Dissolution Test for the Preparations of Examples 11 and 12

Release profiles of the coated core matrix tablet prepared in said Examples 11 and 12 were determined by USP dissolution test apparatus under conditions of simulated intestinal fluid (Fluid II, pH 6.8), paddle type II and 50 rpm/900 ml and time-dependent dissolution level was measured. The result was represented by dissolution percentage as function of time in Table 8.

TABLE 8
Dissolution percentage

| Time (hr) | Example 11 | Example 12 |
|---|---|---|
| 0 | 0.00 | 0.00 |
| 1 | 0.00 | 4.67 |
| 2 | 1.68 | 17.61 |
| 3 | 3.45 | 19.41 |
| 4 | 5.89 | 27.70 |
| 6 | 10.55 | 34.38 |
| 18 | 35.79 | 64.76 |
| 20 | 41.92 | 72.18 |
| 22 | 49.87 | 79.45 |
| 24 | 55.24 | 99.32 |

The dissolution test result for the coated core matrix of Examples 11 and 12 demonstrates the depth of coating and the content of hydrophilic release-modifying polymer, that is, pore-forming material can modify the drug release rate of core matrix showing zero-order release pattern.

### Examples 13˜15

Coated Core Matrix Tablet Containing Oxybutynin

Preparation process for matrix core is the same as in Examples 1-5. Example 13 includes within granules citric acid, substance for regulating pH-surrounding granules, instead of sodium chloride, and includes swelling-regulating material to control the swelling pressure and the swelling speed of matrix. In case of Examples 14 and 15, swelling-regulating material exists in both granules and matrix. As coating substance, shellac was used, and the compositions of the coating solution and the core matrix are represented in the following Table 9.

TABLE 9
Compositions of core matrix tablet containing oxybutynin and coating solution

| | Ingredient (mg) | Example 13 | Example 14 | Example 15 |
|---|---|---|---|---|
| Core Matrix | Oxybutynin hydrochloride | 5 | 5 | 5 |
| | Glyceryl behanate | 15 | 15 | 15 |
| | Dibasic calcium phosphate dihydrate | 28.425 | 28.425 | 28.425 |
| | Lactose | 31.925 | 41.925 | 41.925 |
| | Sodium chloride | — | 17.35 | 17.35 |
| | Citric acid | 17.5 | — | — |
| | Sodium lauryl sulfate | 0.15 | 0.15 | 0.15 |
| | Povidone | 9 | 9 | 9 |
| | Cross-linked sodiumcarboxy methylcellulose | 1.5 | 1.65 | 1.65 |
| | Hydroxypropylmethylcellulose | 30 | 30 | 30 |
| | Magnesium stearate | 1.5 | 1.5 | 1.5 |
| | Moisture* | q.s. | q.s. | q.s. |
| | Total | 150 | 150 | 150 |
| Coating solution | Shellac(OPAGLOSGS-2-0401) | 50% | 50% | 50% |
| | Ethanol | 50% | 50% | 50% |
| | Coating %⁺ | 5 | 1 | 5 |

*Removed during treatment process.
⁺Degree of coating to the weight of uncoated core matrix tablet is shown by %.

### Experimental Example 5

Dissolution Test for the Preparations of Examples 13 and 14

Release profiles of the coated core matrix tablets prepared in said Examples 13 and 14 were determined by USP dissolution test apparatus under conditions of simulated intestinal fluid (Fluid II, pH 6.8), paddle type II and 50 rpm/900 ml and time-dependent dissolution level was measured. The result was represented by dissolution percentage as function of time in Table 10.

TABLE 10
Dissolution percentage

| Time (hr) | Example 13 | Example 14 |
|---|---|---|
| 0 | 0.00 | 0.00 |
| 1 | 1.20 | 3.96 |
| 2 | 3.28 | 9.72 |
| 3 | 22.85 | 24.45 |
| 4 | 30.15 | 32.45 |
| 6 | 43.64 | 40.94 |
| 19 | 79.36 | 86.58 |
| 20 | 81.34 | 90.45 |
| 22 | 84.22 | 93.63 |
| 24 | 87.00 | 98.03 |

The dissolution test result for the coated core matrix tablets of Examples 13 and 14 shows that achieving release-delay effect over a certain amount of time by controlling depth of shellac coating leads to biphasic release pattern. The release-delay and the rapid drug release after the period can be induced by regulating the content of swelling-regulating material contained in the core matrix.

### Experimental Example 6

Dissolution Test for the Preparations of Examples 13˜15

Release profiles of the coated core matrix tablets prepared in said Examples 13 to 15 were determined by USP dissolution test method (paddle type II, 50 rpm/900 ml). According to the stimulated GI method (Gastrointestinal method), the test was conducted in simulated stomach fluid (Fluid I, pH 1.2) for 2 hours and then under simulated intestinal fluid (Fluid II, pH 6.8), time-dependent dissolution level over 24 hours was measured. The result was represented by dissolution percentage as function of time in Table 11.

TABLE 11
Dissolution Percentage

| Time (hr) | Example 13 | Example 14 | Example 15 |
|---|---|---|---|
| 0 | 0.00 | 0.00 | 0.00 |
| 0.5 | 1.97 | 10.29 | 4.78 |
| 1 | 7.02 | 24.50 | 10.03 |
| 1.5 | 15.34 | 33.90 | 20.96 |
| 2 | 20.54 | 44.03 | 28.13 |
| 3 | 28.87 | 51.67 | 41.58 |
| 4 | 35.30 | 55.25 | 40.00 |
| 6 | 46.86 | 62.19 | 47.18 |
| 18 | 73.23 | 89.89 | 85.36 |
| 20 | 76.85 | 92.43 | 85.02 |
| 22 | 81.44 | 94.67 | 86.37 |
| 24 | 83.50 | 96.41 | 91.26 |

### Examples 16-18

Coated Core Matrix Tablet Containing Oxybutynin

Preparation process of matrix core is the same as in Examples 1-5. Example 16 includes swelling-regulating material within granules and matrix to control swelling pressure and swelling speed of matrix. In case of Examples 17 and 18, the content of swelling and erodible polymer within the matrix was increased or reduced, respectively. As coating substance, a mixture of 1:1 ratio of enteric polymer, i.e., hydroxy-propylmethylcellulose phthalate, and shellac was used. Compositions of the coating solution and core matrix are represented in Table 12.

TABLE 12
Compositions of core matrix tablet containing oxybutynin and coating solution

| | Ingredient (mg) | Example 16 | Example 17 | Example 18 |
|---|---|---|---|---|
| Core Matrix | Oxybutynin hydrochloride | 5 | 5 | 5 |
| | Glyceryl behanate | 15 | 15 | 15 |
| | Dibasic calcium phosphate dihydrate | 28.425 | 28.425 | 28.425 |
| | Lactose | 41.925 | 41.925 | 26.925 |
| | Sodium chloride | 17.35 | 17.35 | 17.35 |
| | Citric acid | — | — | — |
| | Sodium lauryl sulfate | 0.15 | 0.15 | 0.15 |
| | Povidone | 9 | 16.5 | 9 |
| | Cross-linked sodiumcarboxy methylcellulose | 1.65 | 1.65 | 1.65 |
| | Hydroxypropylmethyl cellulose | 30 | 22.5 | 45 |
| | Magnesium stearate | 1.5 | 1.5 | 1.5 |
| | Moisture* | q.s. | q.s. | q.s. |
| | Total | 150 mg | 150 mg | 150 mg |
| Coating solution | Shellac (OPAGLOS GS-2-0401) | 2.68% | 2.68% | 2.68% |
| | Hydroxypropylmethyl cellulose phthalate | 2.68% | 2.68% | 2.68% |
| | Methylene chloride | 48.66% | 48.66% | 48.66% |
| | Ethanol | 45.99% | 45.99% | 45.99% |
| | Coating %⁺ | 4 | 4 | 4 |

*Removed during treatment process
⁺Degree of coating to the weight of the uncoated core matrix tablet is shown by %

### Experimental Example 7

Dissolution Test for the Preparation of Examples 16˜18

Release profiles of the coated core matrix tablets prepared in said Examples 16 to 18 were determined by USP dissolution test method (paddle type II, 50 rpm/900 ml), and according to the simulated GI method (Gastrointestinal method). The test was conducted in simulated stomach fluid (Fluid I, pH 1.2) for 2 hours and then under simulated intestinal fluid (Fluid II, pH 6.8), time-dependent dissolution level over 24 hours was measured. The result was represented by dissolution percentage as function of time in Table 13.

TABLE 13
Dissolution Percentage

| Time (hr) | Example 16 | Example 17 | Example 18 |
|---|---|---|---|
| 0 | 0.00 | 0.00 | 0.00 |
| 0.5 | 0.00 | 0.00 | 0.00 |
| 1 | 0.00 | 0.00 | 0.00 |
| 1.5 | 0.00 | 0.00 | 0.00 |
| 2 | 0.00 | 0.00 | 0.00 |
| 3 | 5.01 | 0.00 | 0.00 |
| 4 | 8.55 | 2.29 | 3.31 |
| 6 | 18.51 | 14.52 | 11.09 |
| 8 | 28.50 | 32.33 | 19.86 |
| 18 | 73.27 | 77.65 | 51.32 |
| 20 | 75.66 | 82.15 | 55.05 |
| 22 | 78.63 | 81.52 | 55.15 |
| 24 | 81.87 | 83.72 | 58.58 |

The dissolution test result for the coated core matrix of Examples 14 to 16 represents that pH-dependent release of drug could be corrected by introducing substance with pH dependency into the coating layer, and that drug release was inhibited during the stay in stomach for 2 to 3 hours and, thereafter exhibited zero-order release pattern up to 24 hours.

### Example 19

Coated Core Matrix Tablet Containing Ketorolac

Ketorolac tromethamine, glyceryl behanate, solubilizer, binder, release-regulating material and inert diluents were mixed for 10 minutes at dry state. The mixture, after water was added, was granulated for 5 minutes. The granules thus formed were screened through 18-mesh sieve and dried in an oven at 24 to 40° C. for 12 to 24 hours. The dried granules were screened with 20-mesh sieve. Hydroxypropylmethyl cellulose, binders, swelling-regulating agent and diluents were added to the screened granules, and then they were mixed for 10 minutes. Finally, lubricant was added to them, and then they were mixed for 5 minutes. The mixture was compressed to prepare tablets. Thus prepared core matrix tablets were spray coated in pan coater and dried in oven at 40 to 50° C. for 12 to 24 hours. Table 14 represents the ingredients of the core matrix tablet and composition of the coating solution.

TABLE 14
Composition of the core matrix tablet
and the coating solution

| | Ingredient (mg) | Example 19 |
|---|---|---|
| Core Matrix | Ketorolac tromethamine | 10 |
| | Glyceryl behanate | 30 |
| | Dibasic calcium phosphate dihydrate | 39.35 |
| | Sodium chloride | 15 |
| | Sodium lauryl sulfate | 0.15 |
| | Povidone | 9 |
| | Hydroxypropylmethylcellulose | 45 |
| | Magnesium stearate | 1.5 |
| | Moisture* | q.s. |
| | Total | 150 |
| Coating solution | Hydroxypropylmethylcellulose | 9.6% |
| | Ethyl cellulose | 2.4% |
| | Methylene chloride | 93.4% |
| | Ethanol | 93.4% |
| | Castor oil | 1.2% |
| | Coating %⁺ | 10 |

*Removed during treatment process
⁺Degree of coating to the weight of the uncoated core matrix tablet is shown by %

### Experimental Example 8

Dissolution Test for the Preparations of Example 19

Release profile of the coated core matrix tablet prepared in said Example 17 was determined by USP dissolution test method under condition of simulated intestinal fluid (Fluid II, pH 6.8), paddle type II and 50 rpm/900 ml, and time-dependent dissolution level was measured. The result was represented by dissolution percentage as function of time in Table 15.

TABLE 15
Dissolution percentage

| Time (hr) | Example 19 |
|---|---|
| 0 | 0.00 |
| 1 | 20.61 |
| 2 | 33.43 |
| 3 | 44.80 |
| 4 | 54.33 |
| 6 | 70.26 |
| 8 | 83.40 |
| 12 | 96.17 |

Ketorolac was released from the coated core matrix tablets of Example 19 at a constant rate up to 12 hours, and the release rate could be regulated by the content of swelling material within the matrix and by the coating depth.

### Example 20

Coated Core Matrix Tablet Containing Enalapril Maleate

Therapeutic composition containing enalapril maleate according to the present invention is prepared as follows. First, enalapril maleate, glyceryl behanate, solubilizer, binder, release-regulating substance and inert diluents were mixed for 10 minutes at dry state. The mixture, after water was added, was granulated for 5 minutes. Granules thus formed was screened through 18-mesh sieve and dried in an oven at 24 to 40° C. for 12 to 24 hours. The dried granules were screened with 20-mesh sieve. Hydroxypropylmethylcellulose, binders, swelling-regulating agent and diluents were added to the screened granules, and then they were mixed for 10 minutes. Finally, magnesium stearate was added to them, and then they were mixed for 5 minutes. The mixture was compressed to prepare tablets. Thus prepared core matrix tablets were spray coated in pan coater and dried in oven at 40 to 50° C. for 12 to 24 hours. Table 16 represents the ingredients of the core matrix tablet and composition of the coating solution.

TABLE 16
Compositions of core matrix tablet
and coating solution

| | Ingredient (mg) | Example 20 |
|---|---|---|
| Core Matrix | Enalapril maleate | 10 |
| | Glyceryl behanate | 30 |
| | Dibasic calcium phosphate dihydrate | 39.35 |
| | Sodium chloride | 15 |
| | Sodium lauryl sulfate | 0.15 |
| | Povidone | 9 |
| | Hydroxypropylmethylcellulose | 45 |
| | Magnesium stearate | 1.5 |
| | Moisture* | q.s. |
| | Total | 150 |
| Coating solution | Hydroxypropylmethylcellulose | 9.6% |
| | Ethyl cellulose | 2.4% |
| | Methylene chloride | 93.4% |
| | Ethanol | 93.4% |
| | Castor oil | 1.2% |
| | Coating %⁺ | 10 |

*Removed during treatment process
⁺Degree of coating to the weight of the uncoated core matrix tablet is shown by %

### Experimental Example 9

Dissolution Test for the Preparations of Example 18

Release profile of the coated core matrix tablet prepared in said Example 18 was determined by USP dissolution test method under conditions of simulated intestinal fluid (Fluid II, pH 6.8), paddle type II and 50 rpm/900 ml, and time-dependent dissolution level was measured. The result was represented by dissolution percentage as function of time in Table 17.

TABLE 17
Dissolution percentage

| Time (hr) | Example 18 |
|---|---|
| 0 | 0.00 |
| 1 | 20.61 |
| 2 | 33.43 |
| 3 | 44.80 |
| 4 | 54.33 |
| 6 | 70.26 |
| 8 | 83.40 |
| 12 | 96.17 |

### Example 21

Coated Core Matrix Tablet Containing Captopril

Therapeutic composition containing captopril according to the present invention is prepared as follows. First, captopril, glyceryl behanate, solubilizer, binder, release-regulating substance and inert diluents were mixed for 10 minutes at dry state. The mixture, after water was added, was granulated for 5 minutes. Granules thus formed was screened through 18-mesh sieve and dried in an oven at 24 to 40° C. for 12 to 24 hours. The dried granules were screened with 20-mesh sieve. Hydroxypropylmethylcellulose, binders, swelling-regulating agent and diluents were added to the screened granules, and then they were mixed for 10 minutes. Finally, magnesium stearate was added to them, and then they were mixed for 5 minutes. The mixture was compressed to prepare tablets. Thus prepared core matrix tablets were spray coated in pan coater and dried in oven at 40 to 50° C. for 12 to 24 hours. Ingredients of the core matrix tablet and composition of the coating solution are shown in Table 18.

TABLE 18
Compositions of core matrix tablet
and coating solution

| | Ingredient (mg) | Example 21 |
|---|---|---|
| Core Matrix | Captopril | 25 |
| | Glyceryl behanate | 62.5 |
| | Dibasic calcium phosphate dihydrate | 5 |
| | Povidone | 5 |
| | Hydroxypropylmethylcellulose | 150 |
| | Magnesium stearate | 2.5 |
| | Moisture* | q.s. |
| | Total | 250 |
| Coating solution | Hydroxypropylmethylcellulose | 9.6% |
| | Ethyl cellulose | 2.4% |
| | Methylene chloride | 93.4% |
| | Ethanol | 93.4% |
| | Castor oil | 1.2% |
| | Coating %⁺ | 10 |

*Removed during treatment process
⁺Degree of coating to the weight of the uncoated core matrix tablet is shown by %

### Experimental Example 10

Dissolution Test for the Preparations of Example 21

Release profile of the coated core matrix tablet prepared in Example 19 was determined by USP dissolution test method under conditions of simulated intestinal fluid (Fluid II, pH 6.8), paddle type II and 50 rpm/900 ml, and time-dependent dissolution level was measured. The result was represented by dissolution percentage as function of time in Table 19.

TABLE 19
Dissolution percentage

| Time (hr) | Example 21 |
|---|---|
| 0 | 0.00 |
| 1 | 13.64 |
| 2 | 23.51 |
| 3 | 33.40 |
| 4 | 38.77 |
| 8 | 61.48 |
| 19 | 80.67 |
| 20 | 82.13 |
| 22 | 84.19 |
| 24 | 90.79 |

### Example 22

Preparation of Core Matrix Tablets Containing Diltiazem

Therapeutic composition containing diltiazem according to the present invention is prepared as follows. First, diltiazem hydrochloride, glyceryl behanate, solubilizer, binder, release-regulating substance and inert diluents were mixed for 10 minutes at dry state. The mixture, after water was added, was granulated for 5 minutes. Granules thus formed was screened through 18-mesh sieve and dried in an oven at 24 to 40° C. for 12 to 24 hours. The dried granules were screened with 20-mesh sieve. Hydroxypropylmethylcellulose, binders, swelling-regulating agent and diluents were added to the screened granules, and then they were mixed for 10 minutes. Finally, magnesium stearate was added to them, and then they were mixed for 5 minutes. The mixture was compressed to prepare tablets. Ingredients of the core matrix tablet are shown in Table 20.

TABLE 20
Compositions of core matrix tablet
containing diltiazem

| | Ingredient (mg) | Example 22 |
|---|---|---|
| Core Matrix | Diltiazem hydrochloride | 90 |
| | Glyceryl behanate | 40 |
| | Dibasic calcium phosphate dihydrate | 90 |
| | Sodium chloride | 45 |
| | Sodium lauryl sulfate | 1 |
| | Povidone | 10 |
| | Hydroxypropylmethylcellulose | 120 |
| | Magnesium stearate | 4 |
| | Moisture* | q.s. |
| | Total | 400 |

*Removed during treatment process

### Experimental Example 11

Dissolution Test for the Preparations of Example 22

Release profile of the coated core matrix tablet prepared in said Example 22 was determined by USP dissolution test method under conditions of simulated intestinal fluid (Fluid II, pH 6.8), paddle type II and 50 rpm/900 ml, and time-dependent dissolution level was measured. The result was represented by dissolution percentage as function of time in Table 21.

TABLE 21
Dissolution percentage

| Time (hr) | Example 22 |
|---|---|
| 0 | 0.00 |
| 1 | 13.40 |
| 2 | 20.94 |
| 3 | 27.56 |
| 4 | 33.58 |
| 6 | 45.12 |
| 8 | 55.18 |
| 10 | 64.38 |
| 12 | 72.01 |
| 16 | 90.50 |
| 20 | 100.72 |

### Example 23

Preparation of Core Matrix Tablets Containing Theophylline

Therapeutic composition containing theophylline according to the present invention is prepared as follows. First, theophylline hydrochloride, glyceryl behanate, solubilizer, binder, release-regulating substance and inert diluents were mixed for 10 minutes at dry state. The mixture, after water was added, was granulated for 5 minutes. Granules thus formed was screened through 18-mesh sieve and dried in an oven at 24 to 40° C. for 12 to 24 hours. The dried granules were screened with 20-mesh sieve. Hydroxypropylmethylcellulose, binders, swelling-regulating agent and diluents were added to the screened granules, and then they were mixed for 10 minutes. Finally, magnesium stearate was added to them, and then they were mixed for 5 minutes. The mixture was compressed to prepare tablets. Table 22 gives the ingredients of the core matrix tablet.

TABLE 22
Composition of core matrix tablet
containing theophylline

| | Ingredient (mg) | Example 23 |
|---|---|---|
| Core Matrix | Theophylline | 200 |
| | Glyceryl behanate | 80 |
| | Dibasic calcium phosphate dihydrate | 380 |
| | Sodium chloride | 90 |
| | Sodium lauryl sulfate | 2 |
| | Povidone | 20 |
| | Hydroxypropylmethylcellulose | 120 |
| | Magnesium stearate | 8 |
| | Moisture* | q.s. |
| | Total | 900 |

*Removed during treatment process

### Experimental Example 12

Dissolution Test for the Preparations of Example 23

Release profile of the coated core matrix tablet prepared in said Example 23 was determined by USP dissolution test method under conditions of simulated intestinal fluid (Fluid II, pH 6.8), paddle type II and 50 rpm/900 ml, and time-dependent dissolution level was measured. The result was represented by dissolution percentage as function of time in Table 23.

TABLET 23
Dissolution percentage

| Time (hr) | Example 23 |
|---|---|
| 0 | 0.00 |
| 1 | 11.83 |
| 2 | 17.60 |
| 3 | 22.65 |
| 4 | 26.87 |
| 6 | 35.11 |
| 8 | 41.73 |
| 10 | 47.61 |
| 12 | 50.37 |
| 24 | 72.19 |

The present invention can provide a constant release rate over a period of 8 to 24 hours or more, by allowing drug to be released from granules released from matrix, as well as directly from inside of the matrix, and by regulating the release rate of the granules by the content of swelling-regulating material within the matrix. Further, the present invention minimized solubility-limit of drug by applying a suitable manufacturing method and components of the granules in consideration of water-solubility of drug.

The present invention provides oral drug controlled-release preparation with sustained-release effect proper to the characteristics of drug action, as well as with improved stability, by inducing zero-order release through effectively allowing drug release area to be maintained at a fixed level and through introducing a release-modifying layer.

We claim:

1. A controlled-release oral preparation characterized in that release of granules from matrix and drug release from the granules are conducted in stepwise way, wherein the preparation comprises:
(a) granules comprising a drug and a carrier material in size of 0.1˜1 mm, said carrier material is hydrophobic material in case of drug with water-solubility of 1 mg/ml or more and said carrier material is hydrophilic material in case of drug with water-solubility of less than 1 mg/ml;
(b) a matrix in which the granules are embedded, comprising swelling and erodible polymer and swelling-regulating material; and
(c) a release-modifying layer comprising hydrophobic release-modifying polymer, hydrophilic release-modifying polymer, pH-dependent release-modifying polymer or a mixture thereof.

2. The controlled-release oral preparation in claim 1, wherein 50 to 100% of the drug is present within the granules, and the remaining drug exists within the matrix or the release-modifying layer, or within the matrix and the release-modifying layer in directly dispersed form.

3. The controlled-release oral preparation in claim 1, wherein the drug has a water-solubility within range from 1 mg/ml to 100 mg/ml, and the granules containing the drug is prepared by wet granulation.

4. The controlled-release oral preparation in claim 1, wherein the drug has a water-solubility of at least 100 mg/ml, and the granules containing the drug is prepared in granular form by dispersing the drug in fusion of granules components.

5. The controlled-release oral preparation in claim 1, wherein the drug has a water-solubility of less than 1 mg/ml, and the granules containing the drug is prepared by solid dispersion method.

6. The controlled-release oral preparation in claim 1, wherein the hydrophobic material is at least one selected from the group consisting of fatty acids, fatty acid esters, fatty acid alcohols, fatty acid mono-, di-, tri-glycerides, waxes, hydrogenated castor oil and hydrogenated vegetable oil.

7. The controlled-release oral preparation in claim 6, wherein the fatty acid alcohol is at least one selected from the group consisting of cetostearyl alcohol, stearyl alcohol, lauryl alcohol and myristyl alcohol; fatty acid ester is at least one selected from the group consisting of glyceryl monostearate, glycerol monooleate, acetylated monoglyceride, tristearin, tripalmitin, cetyl ester wax, glyceryl palmitostearate and glyceryl behanate; and wax is at least one selected from the group consisting of beeswax, carnauba wax, glyco wax and castor wax.

8. The controlled-release oral preparation in claim 1, wherein the hydrophilic material is selected from polyalkylene glycol, carboxyvinyl hydrophilic polymer or a mixture thereof, and the drug is solid-dispersed in said hydrophilic polymer.

9. The controlled-release oral preparation in claim 1, wherein the swelling and erodible polymer is at least one selected from the group consisting of hydroxypropyl cellulose, hydroxypropylmethylcellulose, polyethylene oxide, sodium alginate, povidone, polyvinyl alcohol and sodium carboxymethylcellulose.

10. The controlled-release oral preparation in claim 1, wherein said swelling-regulating material is at least one selected from the group consisting of cross-linked sodium carboxymethylcellulose and cross-linked polyvinylpyrrolidone.

11. The controlled-release oral preparation in claim 1, wherein said hydrophobic release-modifying polymer used for the formation of release-modifying layer, is at least one selected from the group consisting of ethylcellulose, shellac and ammonio methacrylate copolymer; said hydrophilic release-modifying polymer is at least one selected from the group consisting of hydroxyalkylcellulose and hydroxypropylalkylcellulose; and said pH-dependent release-modifying polymer is at least one selected from the group consisting of hydroxyalkylcellulose phthalate, hydroxyalkylmethylcellulose phthalate, cellulose acetyl phthalate, sodium cellulose acetate phthalate, cellulose ester phthalate, cellulose ether phthalate, and anionic copolymer of methacrylic acid with methyl or ethyl methacrylate.

12. The controlled-release oral preparation in claim 1, wherein said release-modifying layer is 1 to 20% by weight to total weight of matrix, and the granules containing the drug reach 50 to 80% by weight to total weight of the preparation.

13. The controlled-release oral preparation in claim 1, wherein the drug is selected from:
therapeutic agents for aconuresis of oxybutynin, tolterodine and therapeutically equivalent salts thereof;
calcium channel blockers of nifedipine, verapamil, isradipin, nilvadipin, flunarizine, nimodipine, diltiazem, nicardipine, nisoldipin, felodipin, amlodipin, cinarizin and pendilin and pharmaceutically acceptable derivatives thereof;
beta adrenergic antagonists of propranolol, metoprolol and pharmaceutically acceptable derivatives thereof;
angiotensin-converting enzyme inhibitors of captopril, enalapril, ramipril, fosinopril, altiopril, benazepril, libenzapril, alacepril, cilazapril, cilazaprilat, perindopril, zofedopril, lisinopril, imidapril, spirapril, rentiapril, delapril, alindapril, indalapril, quinalapril and therapeutically equivalent salts thereof;
non-steroidal anti-inflammatory agents of ketorolac, ketoprofen, benoxaprofen, caprofen, flubiprofen, fenoprofen, suprofen, fenbufen, ibuprofen, indoprofen, naproxen, miroprofen, oxaprozine, pranoprofen, pirprofen, thiaprofenic acid, fluprofen, alminoprofen, bucloxic acid, alclofenac acematacin, aspirin, indomethacin, ibufenac, isoxepac, profenac, fentiazac, clidanac, oxpinac, sulindac, tolmetin, zomepirac, zidometacin, tenclofenac, tiopinac, mefenamic acid, flufenamic acid, niflumic acid, meclofenamic acid, tolfenamic acid, diflufenisal, isoxicam, sudoxicam and therapeutically equivalent salts thereof;
therapeutic agents for respiratory disorders of theophylline, salbutamol, aminophylline, dextromethorphan, pseudoephedrine and therapeutically equivalent salts thereof;
analgesics of tramadol, acetaminophen, morphine, hydromorphone, oxycodone, propoxyphene and therapeutically equivalent salts thereof;
psychoneural drugs of fluoxetine, paroxetine, buspirone, carmabazepine, carvidopa, levodopa, methylphenidate, trazodone, valproic acid, amitriptyline, carbamazepine, ergoloid, haloperidol, lorazepam and therapeutically equivalent salts thereof;
antibiotics of azithromycin dihydrate, cepha antibiotics, clarithromycin, doxycycline, nitrofurantonin and therapeutically equivalent salts thereof;
antihyperlipidemic agent of bezafibrate, fenofibrate, ethofibrate, lovastatin and therapeutically equivalent salts thereof;
antidiabetic agent of glyburide, glipizide, metformin and therapeutically equivalent salts thereof; and
cyclobenzaprin, favotidin, nizatidine, propafenone, clonazepam, hyoscyamine, diphenhydramine, olistat, doxazosin and therapeutically equivalent salts thereof.

14. The controlled-release oral preparation in claim 1, wherein the drug is released in zero-order over at least 8 to 24 hr upon the administration of the preparation.

15. The controlled-release oral preparation in claim 1, wherein by erosion of the surface of matrix, 0 to 20% of total granules is released over 0 to 4 hr, 0 to 50% is released over 0 to 8 hr, 0 to 70% is released over 0 to 16 hr, and 0 to 100% is released over 0 to 24 hours.
